(19) United States
(12) Patent Application Publication
(10) Pub. No.: US 2026/0090887 A1
(43) Pub. Date: Apr. 2, 2026

(54) Epicardial device and methods for right heart support during LVAD and cardiac procedures

(71) Applicant: John M. MacMahon, Exeter, NH (US)

(72) Inventor: John M. MacMahon, Exeter, NH (US)

(21) Appl. No.: 18/831,793

(22) Filed: Sep. 15, 2025

Related U.S. Application Data
(60) Provisional application No. 63/694,747, filed on Sep. 13, 2024.

(51) Int. Cl. A61F 2/24; A61F 2/48; A61M 60/178

(52) U.S. Cl. CPC A61F 2/2481; A61F 2/2442; A61F 2/482; A61M 60/178; A61F 2220/0008; A61F 2250/0001

(57) ABSTRACT

An epicardial implant system prevents right heart failure following left ventricular assist device (LVAD) implantation through dual-mechanism support of the right ventricular free wall and tricuspid valve annulus. The device comprises a biocompatible independent adjustment mechanisms for ventricular wall constraint and annular geometry optimization. The invention simultaneously treats both ventricular dilation and annular valve dysfunction through separate adjustable elements. Epicardial placement eliminates atriotomy requirements while providing real-time hemodynamic optimization. Atraumatic anchoring permits repositioning without tissue damage. Preclinical validation confirms six-minute implantation with independent component efficacy under afterload conditions. The ventricular component prevents dilation while the annular component reduces regurgitation, validating dual-mechanism necessity. The prophylactic approach addresses mechanical disruption causing right heart failure in 40% of LVAD recipients. Applications extend beyond LVAD to coronary bypass, and valvular corrections. Biodegradable embodiments accommodate pediatric cardiac growth. The system provides the first dedicated prophylactic intervention for post-surgical right heart failure across multiple cardiac procedures.

### CROSS-REFERENCE TO RELATED APPLICATIONS

This application claims the benefit of U.S. Provisional Patent Application No. 63/694,747 , filed Sep. 13, 2024, which is incorporated herein by reference in its entirety.

### STATEMENT REGARDING FEDERALLY SPONSORED RESEARCH OR DEVELOPMENT

None. No federally sponsored research or development was involved in the conception or reduction to practice of the inventions described herein.

### THE NAMES OF THE PARTIES TO A JOINT RESEARCH AGREEMENT

None. No joint research agreement was involved in the conception or reduction to practice of the inventions described herein.

### INCORPORATION-BY-REFERENCE

Not applicable

### STATEMENT REGARDING PRIOR DISCLOSURES BY THE INVENTOR OR A JOINT INVENTOR

The inventor, John MacMahon, presented aspects of this invention at the Octane Cardiovascular Tech Forum on September 13-14, 2024. This disclosure was made by the inventor less than one year prior to the filing date of this application.

### BACKGROUND OF THE INVENTION

Left Ventricular Assist Device (LVAD) implantation is an essential therapy for patients with advanced left-sided heart failure, providing significant improvement in cardiac output and survival. However, this intervention fundamentally alters intracardiac hemodynamics, breaking the physiologic balance between the right and left sides of the heart. Acute increases in left-sided flow imposed by the LVAD create a critical overload on the right side, with right-sided heart failure (RHF) developing in up to 40% of recipients following device implantation. Complications of RHF, including re-operations, prolonged hospital stays, and persistent functional disability, have rendered RHF the leading cause of mortality and morbidity after LVAD surgery.

Despite these high stakes, effective preventative strategies for RHF remain conspicuously absent. Surgeons currently lack dedicated devices for preserving right heart geometry and valve competence after LVAD implantation. The acute and chronic mechanical overload of the right ventricle (RV) manifests as dilation and annular distortion, which in turn precipitate tricuspid regurgitation and progressive ventricular dysfunction. Notably, traditional repair techniques, including tricuspid annuloplasty and valve repair, focus solely on annular reshaping but fail to address the free wall dilation that is a key driver of post-LVAD RV failure. Moreover, these surgical approaches can introduce additional risks, such as bleeding complications or atrial injury, and have a high failure rate in the setting of ongoing mechanical stress.

Existing epicardial restraint devices have historically been designed for left ventricular remodeling or global heart support, and are inadequate for targeted RV intervention. Screening tools for predicting RHF have limited sensitivity, and late RHF (occurring after 30 days post-LVAD) is particularly lethal, with reported two-year mortality rates exceeding 80% once diagnosed. As documented in the MOMENTUM 3 trial, LVAD-induced right heart failure continues to affect approximately 28-34% of patients with centrifugal-flow pumps despite technological advances in device design. As a result, late RV dysfunction continues to erode advances made in LVAD engineering and patient management.

There is, therefore, an urgent unmet clinical need for a prophylactic, adjustable device that directly counteracts the abnormal mechanical forces imposed on the right heart by LVADs. Such a device must provide independent, surgeon-controlled support to both the RV free wall and tricuspid valve annulus, restoring and preserving right-sided heart structure and function immediately upon LVAD activation. Meeting this need would proactively protect patients from RHF, reduce the incidence of rehospitalization, and improve both short-term and long-term survival following mechanical circulatory support.

While LVAD-induced RHF represents the most acute manifestation of this problem, right heart dysfunction is a broader challenge across multiple cardiac surgical procedures. Patients undergoing coronary artery bypass surgery, valve replacement procedures, and other mechanical circulatory support interventions frequently experience similar patterns of right heart stress and failure. Cardiac surgery patients with pre-existing pulmonary hypertension, ischemic heart disease, or dilated cardiomyopathy are particularly vulnerable to post-operative right heart decompensation. Additionally, patients with congenital heart disease undergoing corrective surgery often exhibit right heart dysfunction as a primary or secondary consequence of their underlying condition and surgical intervention. The mechanical principles underlying RV free wall dilation and tricuspid valve incompetence remain consistent across these various clinical scenarios, creating a common pathophysiologic pathway that current therapeutic approaches fail to adequately address.

There is, therefore, an urgent unmet clinical need for a prophylactic, adjustable device that directly counteracts abnormal mechanical forces imposed on the right heart across a spectrum of cardiac surgical procedures. Such a device must provide independent, surgeon-controlled support to both the RV free wall and tricuspid valve annulus, restoring and preserving right-sided heart structure and function. Meeting this need would proactively protect patients from RHF across multiple cardiac surgery contexts, reduce the incidence of rehospitalization, and improve both short-term and long-term survival following cardiac interventions.

### FIELD OF THE INVENTION

The present invention relates to medical devices for the treatment of right heart failure and tricuspid valve dysfunction in patients undergoing cardiac surgery procedures, and more particularly to an epicardial implant system that provides independent, adjustable support to both the right ventricular free wall and tricuspid valve annulus to prevent and treat cardiac surgery-induced right heart failure, with particular application to patients receiving left ventricular assist device (LVAD) support and other mechanical circulatory support systems.

### Description of Related Art

The following description of related art includes references to prior patents, published patent applications, and non-patent literature that may be relevant to the present invention, as disclosed in accordance with 37 CFR 1.97 and 1.98.

The following description of related art includes references to prior patents, published patent applications, and non-patent literature that may be relevant to the present invention, as disclosed in accordance with 37 CFR 1.97 and 1.98.

Several prior art epicardial devices have been developed for cardiac applications, primarily focused on left ventricular remodeling. U.S. Pat. No. 6,221,103 to Melvin discloses a geometric reconfiguration assembly for restraining heart chamber geometry using a collar with multiple bands and connector bars. While this device addresses ventricular dilation, it is designed for global heart restraint rather than targeted right heart intervention and lacks the independent adjustability required for LVAD-specific applications.

U.S. Pat. No. 7,247,134 to Vidlund et al. describes devices and methods for heart valve treatment using epicardial force application. However, these devices focus primarily on mitral valve applications and do not address the specific mechanical challenges of right heart failure or the dual pathology of RV free wall dilation combined with tricuspid valve dysfunction.

U.S. Pat. No. 11,058,540 to MacMahon et al. discloses an atraumatic adjustment system for epicardial valve repair devices. While this patent demonstrates adjustable epicardial approaches to valve therapy, it is specifically designed for mitral valve applications and does not address right heart pathology or the unique hemodynamic challenges associated with LVAD support.

International Application WO 2019/148046 A1 also to MacMahon et al. describes an epicardial valve repair system with multi-component delivery. This system, while innovative in its delivery approach, is similarly focused on left-sided heart applications and lacks the specific design features necessary for right heart intervention.

U.S. Patent Application 2008/0109076 A1 to Cartledge et al. describes methods for controlling internal circumference of anatomic orifices, including cardiac valve applications. While this approach addresses annular control, it relies on intracardiac placement and does not provide the epicardial free wall support necessary to address the mechanical etiology of LVAD-induced RHF.

Current clinical literature, including recent meta-analyses such as Luo et al. (2024), demonstrates that conventional tricuspid valve interventions during LVAD implantation have limited efficacy and may actually increase the risk of right heart failure. These studies consistently show that annuloplasty alone fails to prevent late RHF and does not address the fundamental problem of RV free wall dilation.

The MOMENTUM 3 trial and subsequent analyses have documented the persistent problem of LVAD-induced right heart failure, affecting 28-34% of patients despite advances in device technology. Current management strategies are largely reactive, focusing on inotropic support, temporary mechanical support, or valve repair after RHF has already developed.

As documented in SBIR grant applications and clinical reviews, existing approaches fail to address the fundamental mechanical disruption caused by LVAD support. The literature consistently identifies the need for prophylactic interventions that can address both RV dilation and tricuspid valve competence simultaneously.

Current surgical approaches to tricuspid valve dysfunction typically require atriotomy (opening of the right atrium) to gain direct access to the valve structures, necessitating cardiopulmonary bypass and cardiac arrest. These invasive procedures carry substantial risks including bleeding complications from atrial suture lines, air embolism, conduction system damage, and the morbidity associated with cardiopulmonary bypass itself. Additionally, atriotomy-based repairs often fail to address the underlying right ventricular free wall pathology that drives tricuspid regurgitation, focusing solely on annular reconstruction while leaving the mechanical forces causing valve dysfunction unaddressed. The requirement for cardiac arrest and bypass support significantly increases operative time, resource utilization, and patient risk, particularly in the already compromised LVAD patient population. Furthermore, the inability to assess valve function and right heart geometry under physiologic conditions during atriotomy-based procedures limits the surgeon's ability to optimize repair effectiveness, often resulting in suboptimal outcomes that become apparent only after weaning from bypass when revision is no longer feasible without repeat sternotomy and bypass support.

The prior art suffers from several critical limitations when applied to LVAD-induced right heart failure. Existing epicardial devices are designed for left heart applications and do not conform to right heart anatomy or address right heart-specific pathophysiology. Current interventions address either valve function or ventricular geometry, but not both simultaneously, failing to address the dual pathology of LVAD-induced RHF. Most existing devices provide static support without the ability to adjust for the dynamic changes that occur after LVAD activation. Existing approaches are reactive rather than prophylactic, attempting to treat RHF after it has developed rather than preventing its occurrence. Published clinical data consistently demonstrates poor outcomes with current interventions, with high rates of persistent or recurrent RHF.

None of the prior art teaches or suggests an epicardial device specifically designed for right heart applications that provides independent, adjustable support to both the RV free wall and tricuspid valve annulus. The present invention addresses this gap by providing a prophylactic, adjustable system specifically designed for the unique mechanical challenges of LVAD-induced right heart failure while being broadly applicable to other cardiac surgery procedures involving right heart dysfunction.

### BRIEF SUMMARY OF THE INVENTION

The present invention provides a novel epicardial implant system specifically designed to prevent and treat right heart failure associated with cardiac surgery procedures, with particular efficacy in addressing LVAD-induced right heart failure. The invention recognizes that current approaches fail because they address only single aspects of a dual pathology—right ventricular free wall dilation and tricuspid valve dysfunction—and provides the first device capable of independently supporting both anatomical structures simultaneously.

The FreeWall epicardial implant comprises a biocompatible support framework, preferably incorporating titanium, silicone, and polyester components, configured for placement on the epicardial surface of the right heart. The device features independent adjustment mechanisms that allow surgeons to optimize support for both the right ventricular free wall and the tricuspid valve annulus according to individual patient anatomy and hemodynamic requirements.

Unlike prior art devices that provide static, global heart restraint, the present invention enables precise, localized intervention with real-time adjustability during surgical procedures. The device incorporates patient-specific sizing options and intraoperative adjustment capabilities, allowing surgeons to optimize therapeutic effect while minimizing adverse impact on cardiac function.

Preclinical validation in four porcine studies has demonstrated the practical feasibility and clinical efficacy of the FreeWall implant. Prototype devices achieved successful implantation in under six minutes, demonstrating the surgical efficiency of the system. The independent adjustability feature was validated through controlled studies where pulmonary restriction simulated increased afterload conditions. Under these challenging hemodynamic conditions, the annular component of the implant successfully reduced the induced tricuspid regurgitation when placed under appropriate tension, and tricuspid regurgitation was restored to baseline levels when the device tension was reduced, confirming the controllable and reversible nature of the therapeutic effect.

Critically, the ventricular component provided independent protection from induced afterload dilation, demonstrating that ventricular wall support alone contributes significantly to right heart function preservation. When the ventricular component was relaxed, right ventricular dilation occurred and tricuspid regurgitation was impacted, validating that right heart failure has dual components and that optimizing treatment and prevention requires a biomechanically matched dual approach addressing both ventricular geometry and valve function simultaneously.

The precision of surgical control was demonstrated using devices with incremental one-millimeter adjustment steps, providing surgeons with excellent fine-tuning capability for targeted support of both ventricular and annular components. All titanium anchors remained securely in place throughout the studies and were removable without blood loss, consistent with previous preclinical and clinical experience using identical anchoring systems for left ventricular applications.

The invention includes several critical components working in concert: a biocompatible epicardial support structure configured for stable, long-term placement on the right ventricular free wall; a tricuspid annular support component that provides adjustable constraint to the tricuspid valve annulus without interfering with leaflet mobility; independent adjustment mechanisms that allow separate optimization of ventricular wall support and annular geometry; and anchoring systems designed for secure, atraumatic attachment to cardiac tissue.

The support framework utilizes materials with proven biocompatibility in cardiac applications, including titanium components for structural integrity and polyester elements for tissue integration. The anchoring system distributes mechanical loads to minimize tissue trauma while providing secure, long-term fixation, as validated by the successful preclinical anchor performance.

A fundamental innovation of the present invention is its prophylactic rather than reactive approach to right heart failure. The device is designed for implantation concurrent with LVAD placement or other cardiac procedures, providing immediate protection against the mechanical forces that would otherwise lead to right heart decompensation. This represents a paradigm shift from current practice, which attempts to treat right heart failure after it has developed, typically with poor results.

The invention addresses the fundamental limitation of prior art by providing independent support to both components of post-surgical right heart failure: ventricular dilation and valve dysfunction. The preclinical validation confirms that both components must be addressed simultaneously for optimal therapeutic effect, as demonstrated by the independent protective effects of ventricular wall support and the interdependent relationship between ventricular geometry and valve competence. The device enables surgeons to adjust ventricular wall support independently of tricuspid annular constraint, allowing optimization of both therapeutic targets according to individual patient needs.

While particularly effective for LVAD-induced right heart failure, the invention has broad applicability across cardiac surgery procedures. Patients undergoing coronary artery bypass surgery, valve replacement, other mechanical circulatory support interventions, and congenital heart disease corrections frequently experience right heart dysfunction through similar mechanical pathways. The device's adjustable, dual-support design addresses these common pathophysiologic mechanisms across multiple clinical scenarios.

The invention includes surgical methods for device implementation, featuring techniques for optimal placement timing, anatomical positioning, and intraoperative adjustment. The method encompasses preoperative sizing based on imaging studies, surgical placement techniques that minimize procedural complexity, and post-implantation adjustment protocols that optimize long-term outcomes.

Implementation of the present invention provides multiple clinical benefits including prevention of right heart failure rather than reactive treatment, reduction in post-operative complications, rehospitalizations, and revision procedures improved long-term survival and functional outcomes, reduced healthcare costs associated with right heart failure management, and expanded eligibility for advanced heart failure therapies through improved right heart function preservation.

The invention encompasses both apparatus and method claims, including various embodiments of the device structure, alternative anchoring mechanisms, different adjustment systems, and multiple surgical implementation approaches. The scope includes both primary applications in LVAD recipients and broader applications across cardiac surgery procedures involving right heart dysfunction risk.

This comprehensive approach to right heart failure prevention represents a significant advancement in cardiac surgery, providing surgeons with the first dedicated tool for addressing the dual pathology underlying post-surgical right heart failure while maintaining the flexibility to adapt to individual patient anatomy and surgical requirements, as validated through successful preclinical demonstration of both independent component efficacy and synergistic dual-component therapeutic benefit.

### BRIEF DESCRIPTION OF THE SEVERAL VIEWS OF THE DRAWING(S)

FIG. 1 shows a flowchart illustrating the method of implementing the FreeWall epicardial implant system during LVAD implantation, showing the sequential procedural steps from initial cardiac access through dynamic post-activation optimization.

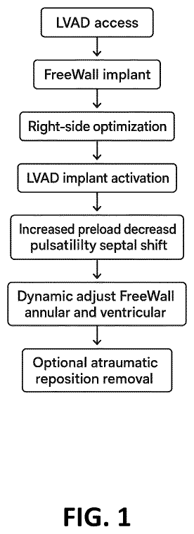

FIG. 2 shows a comparison view of tricuspid valve annular geometry showing (A) normal triangular valve configuration and (B) pathologic annular dilation with pronounced expansion toward the right ventricular free wall region, illustrating the biomechanical basis for the dual-support approach required by the present invention.

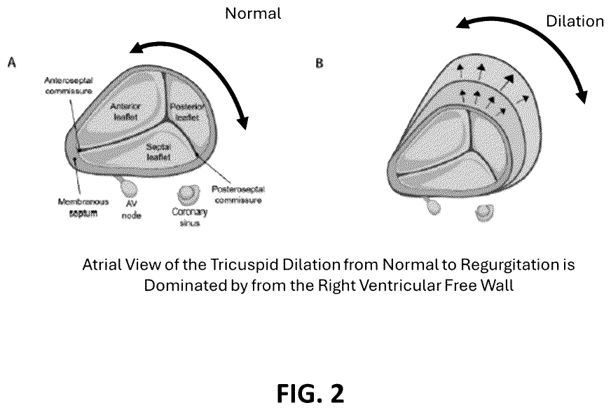

FIG. 3 shows a comparison view of tricuspid valve annular geometry showing (A) normal triangular valve configuration and (B) pathologic annular dilation with pronounced expansion toward the right ventricular free wall region, illustrating the biomechanical basis for the dual-support approach required by the present invention.

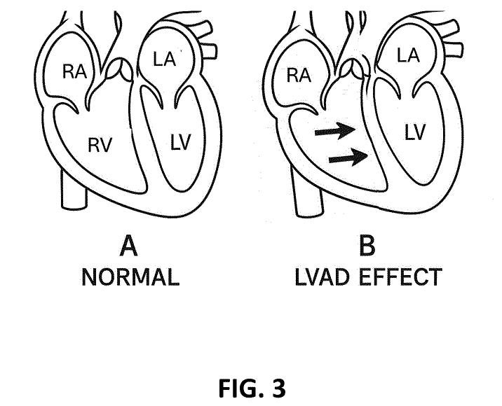

FIG. 4 is the Freewall with both annular and ventricular components on the Right ventricle.

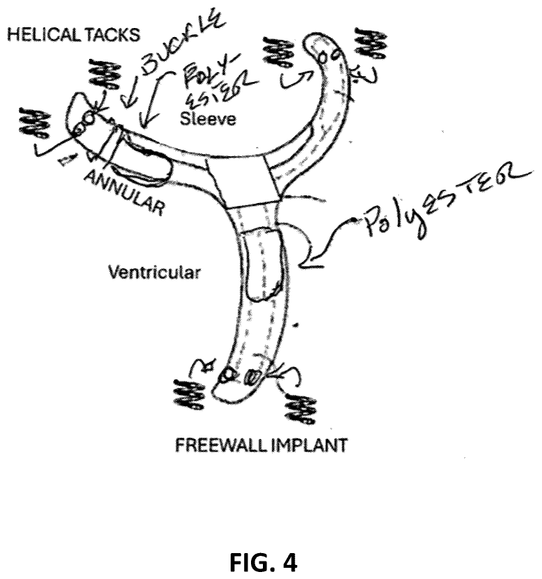

### DETAILED DESCRIPTION OF THE INVENTION

The present invention will now be described more fully hereinafter with reference to the accompanying drawings, in which preferred embodiments of the invention are shown. The invention may, however, be embodied in many different forms and should not be construed as limited to the embodiments set forth herein. Rather, these embodiments are provided so that this disclosure will be thorough and complete, and will fully convey the scope of the invention to those skilled in the art.

The FreeWall epicardial implant system addresses the fundamental problem of right heart failure following cardiac surgery procedures, particularly those involving left ventricular assist device (LVAD) implantation. The device recognizes that right heart failure results from a dual pathology requiring simultaneous intervention: right ventricular free wall dilation and tricuspid valve dysfunction. Unlike prior art approaches that address only single components of this dual pathology, the present invention provides independent, adjustable support to both anatomical structures.

As illustrated schematically in the drawings, LVAD implantation fundamentally alters cardiac hemodynamics by unloading the left ventricle while imposing increased mechanical stress on the right ventricle. This hemodynamic shift results in right ventricular dilation and tricuspid valve incompetence, leading to right heart failure in 10-40% of LVAD recipients. The FreeWall device is specifically designed to counteract these mechanical forces prophylactically, providing immediate protection upon LVAD activation.

The FreeWall epicardial implant comprises several integrated components working in concert to provide dual-mechanism support. The primary structural framework consists of a biocompatible support structure configured for epicardial placement on the right ventricular free wall. This framework preferably incorporates titanium components for structural integrity and polyester elements for tissue integration and biocompatibility.

The ventricular support component is configured to apply controlled constraining forces to the right ventricular free wall, preventing the pathologic dilation that occurs following LVAD implantation. The component is designed with patient-specific sizing options and incorporates adjustment mechanisms that allow surgeons to optimize the degree of ventricular constraint according to individual anatomy and hemodynamic requirements.

The tricuspid annular support component provides independent constraint to the tricuspid valve annulus without interfering with normal leaflet mobility. This component addresses the annular dilation and geometric distortion that contributes to tricuspid regurgitation in post-LVAD patients. The annular support mechanism is independently adjustable, allowing separate optimization from the ventricular wall support.

The adjustment mechanisms enable precise control over both ventricular wall constraint and annular geometry. In preferred embodiments, adjustment increments of one millimeter provide surgeons with excellent fine-tuning capability for targeted support. The adjustment mechanisms may be operated both during initial implantation and in post-implantation procedures if therapeutic optimization is required.

The anchoring system distributes mechanical loads across multiple discrete attachment points to minimize tissue trauma while providing secure, long-term fixation. Titanium anchors are preferably employed, consistent with proven biocompatible materials used successfully in cardiac applications. The anchoring system is designed for atraumatic placement and, if necessary, removal without significant tissue damage or blood loss.

The support framework utilizes materials with established biocompatibility in long-term cardiac implant applications. Titanium components provide structural integrity while exhibiting excellent corrosion resistance and tissue compatibility. The titanium framework is preferably fabricated from commercially pure titanium or titanium alloys such as Ti-6AI-4V, which provide optimal strength-to-weight ratios and proven biocompatibility.

Polyester components facilitate tissue integration and provide conformable interfaces with cardiac tissue. The polyester elements may comprise woven or knitted fabrics that promote controlled tissue ingrowth while maintaining device stability. Polyethylene terephthalate (PET) fabrics, such as those marketed under the Dacron tradename, are particularly suitable for these applications.

Alternative biocompatible materials may be employed while maintaining the functional characteristics of the device. These may include other biocompatible metals such as stainless steel alloys, cobalt-chromium alloys, or shape-memory alloys such as nitinol. Polymer components may alternatively comprise polytetrafluoroethylene (PTFE), expanded PTFE (ePTFE), or other biocompatible polymers with appropriate mechanical properties.

The FreeWall device is designed for implantation concurrent with LVAD placement or other cardiac procedures requiring right heart protection. The prophylactic approach provides immediate protection against mechanical forces that would otherwise lead to right heart decompensation, representing a paradigm shift from reactive treatment approaches.

An exemplary Freewall annular component with dual anchor silos can extend the distance of the Right Ventricle Freewall, with the helical tacks as an example of its fixation. The compliant component is polyester and the anchors are titanium in this example.

An exemplary Freewall Ventriaular component has a connecting sleeve that slides over the annular component and extends to the apical direction of the ventricle and has dual anchor silos.

Preoperative sizing is preferably performed using standard cardiac imaging techniques including echocardiography, cardiac MRI, or CT angiography. Patient-specific measurements of right ventricular dimensions, tricuspid annular diameter, and anatomical relationships guide device selection from a range of sizes designed to accommodate normal anatomical variation.

Surgical access is typically achieved through median sternotomy or lateral thoracotomy, depending on the primary procedure being performed. The pericardium is opened to provide access to the epicardial surface of the right heart. In minimally invasive procedures, thoracoscopic or robotic approaches may be employed using appropriately sized access ports.

Device placement begins with positioning of the ventricular support component on the right ventricular free wall. The component is positioned to provide optimal constraint against pathologic dilation while avoiding interference with normal cardiac conduction pathways or coronary vascular structures. Titanium anchors are placed through discrete attachment points to secure the ventricular component to the epicardial surface.

The tricuspid annular support component is positioned to provide circumferential constraint around the tricuspid valve annulus. Care is taken to avoid interference with normal valve leaflet motion while providing sufficient constraint to prevent pathologic annular dilation. The annular component is secured using discrete anchoring points distributed around the annular circumference.

Independent adjustment mechanisms allow optimization of both ventricular wall constraint and annular geometry according to individual patient requirements. Initial adjustment is performed during the implantation procedure based on direct visualization and hemodynamic monitoring. Post-implantation adjustments may be performed if clinical conditions warrant therapeutic modification.

Extensive preclinical validation has been performed in four porcine studies demonstrating both the feasibility and efficacy of the FreeWall implant system. These studies validated key performance characteristics including surgical implantation efficiency, independent component functionality, and safety profiles.

Implantation procedures were consistently completed in under six minutes, demonstrating the surgical efficiency of the device and its compatibility with complex cardiac procedures. The rapid implantation time minimizes additional procedural risk and facilitates integration with standard LVAD implantation protocols.

Independent adjustability was validated through preclinical acute porcine studies simulating increased afterload conditions using pulmonary restriction. Under these challenging hemodynamic conditions, the annular component successfully reduced induced tricuspid regurgitation when placed under appropriate tension. Importantly, tricuspid regurgitation returned to baseline levels when device tension was reduced, confirming the controllable and reversible nature of the therapeutic effect.

The ventricular component demonstrated independent protective efficacy against induced afterload dilation. When the ventricular component was appropriately tensioned, right ventricular dilation was prevented despite increased afterload conditions. Conversely, when the ventricular component was relaxed, right ventricular dilation occurred and tricuspid regurgitation was adversely impacted, validating that right heart failure comprises dual components requiring simultaneous biomechanical intervention.

Precision of surgical control was demonstrated using devices with incremental one-millimeter adjustment steps. This level of control provides surgeons with excellent fine-tuning capability for targeted support of both ventricular and annular components according to individual patient anatomy and physiologic requirements.

All titanium anchors remained securely in place throughout the preclinical studies, demonstrating the reliability of the anchoring system under physiologic loading conditions. Importantly, anchors were removable without blood loss when removal was attempted, consistent with previous preclinical and clinical experience using identical anchoring systems for left ventricular applications.

While particularly effective for LVAD-induced right heart failure, the FreeWall device has broad applicability across cardiac surgery procedures. The fundamental mechanical principles underlying right heart dysfunction remain consistent across various clinical scenarios, creating common pathophysiologic pathways that the device is designed to address.

Patients undergoing coronary artery bypass surgery frequently experience right heart stress, particularly those with pre-existing pulmonary hypertension or compromised right ventricular function. The FreeWall device provides prophylactic protection against post-operative right heart decompensation in these high-risk populations.

Valve replacement procedures, particularly those involving mitral or aortic valves, may alter cardiac loading conditions and predispose to right heart dysfunction. The device provides protective support during the post-operative adaptation period when hemodynamic changes may stress right heart function.

Other mechanical circulatory support interventions, including temporary ventricular assist devices, extracorporeal membrane oxygenation (ECMO), and total artificial hearts, create similar hemodynamic alterations that may compromise right heart function. The FreeWall device provides protective support across these various mechanical support modalities.

Congenital heart disease corrections often involve complex hemodynamic alterations that may impact right heart function either as a primary consequence of the underlying condition or secondary to surgical intervention. The adjustable, dual-support design addresses the variable anatomical and physiologic requirements of these diverse patient populations.

Various alternative embodiments of the FreeWall device may be constructed while maintaining the core functional characteristics of dual-mechanism right heart support. These alternatives may incorporate different materials, attachment mechanisms, adjustment systems, or geometric configurations while preserving the essential technical features.

Alternative anchoring systems may employ different biocompatible fixation methods including surgical sutures, tissue adhesives, or mechanical fasteners. The specific anchoring method may be selected based on surgical preference, anatomical considerations, or procedural requirements.

Adjustment mechanisms may incorporate various mechanical, hydraulic, or pneumatic systems to provide the required precision and range of adjustment. Electronic or motorized adjustment systems may be employed for applications requiring remote or automated control.

Geometric configurations may be modified to accommodate specific anatomical variants or procedural requirements. The device may be fabricated in various sizes and shapes to match normal anatomical variation or to address specific pathologic conditions.

Material compositions may be varied while maintaining biocompatibility and mechanical performance requirements. Alternative metal alloys, polymer formulations, or composite materials may be employed based on specific performance requirements or manufacturing considerations.

The FreeWall device is manufactured using established medical device fabrication techniques ensuring consistent quality and performance characteristics. Titanium components are preferably fabricated using precision machining or metal forming techniques that maintain dimensional accuracy and surface finish requirements.

Polyester components are fabricated using established textile manufacturing techniques including weaving, knitting, or non-woven processes. Quality control measures ensure consistent porosity, strength, and biocompatibility characteristics across production lots.

Assembly processes incorporate appropriate quality control measures including dimensional verification, material property testing, and biocompatibility validation. Sterilization is performed using established medical device sterilization methods such as gamma irradiation or ethylene oxide processing.

The FreeWall device is designed to meet applicable regulatory requirements for implantable cardiac devices. Biocompatibility testing follows established standards including ISO 10993 series requirements for biological evaluation of medical devices.

Safety considerations include appropriate material selection, design validation, and clinical testing protocols. The device design incorporates safety features to minimize risks associated with implantation, long-term implantation, and potential device removal if required.

Clinical testing protocols follow established guidelines for investigational cardiac devices, including appropriate preclinical validation, clinical trial design, and regulatory submission requirements.

Implementation of the FreeWall device provides multiple clinical and economic advantages compared to current approaches to right heart failure management. The prophylactic approach prevents right heart failure rather than attempting reactive treatment after dysfunction has developed, typically with superior outcomes and reduced morbidity.

Clinical benefits include reduced post-operative complications, decreased rehospitalization rates, improved long-term survival, and enhanced functional outcomes. Economic benefits include reduced healthcare costs associated with right heart failure management and decreased resource utilization for revision procedures.

The device expands eligibility for advanced heart failure therapies by improving right heart function preservation, potentially enabling more patients to benefit from mechanical circulatory support or cardiac transplantation.

The comprehensive approach to right heart failure prevention represents a significant advancement in cardiac surgery, providing surgeons with the first dedicated tool for addressing the dual pathology underlying post-surgical right heart failure while maintaining the flexibility to adapt to individual patient anatomy and surgical requirements.

This detailed description enables those skilled in the art to make and use the invention and represents the best mode known to the inventor for carrying out the invention. Various modifications and alternative embodiments will be apparent to those skilled in the art while remaining within the scope of the invention as defined by the appended claims.

1. A medical device for preventing right heart failure in patients receiving left ventricular assist device support, comprising:
(a) a biocompatible epicardial support structure configured for placement on right ventricular cardiac tissue;
(b) a right ventricular free wall support component configured to prevent right ventricular dilation;
(c) a tricuspid annular constraint component configured to maintain tricuspid valve geometry and prevent tricuspid regurgitation;
(d) an adjustment mechanism configured to independently modify support parameters of said right ventricular free wall support component and said tricuspid annular constraint component;
(e) anchoring means for securing the device to cardiac tissue during left ventricular assist device implantation; and
(f) wherein the device is configured to address both right ventricular dilation and tricuspid regurgitation simultaneously and independently to prevent LVAD-induced right heart failure.

2. The medical device of claim 1, wherein the right ventricular free wall support component comprises:
(a) a conforming support structure configured to contact the epicardial surface of the right ventricular free wall;
(b) a constraint mechanism configured to limit right ventricular free wall expansion; and
(c) wherein the support structure is positioned to prevent the common area of right ventricular dilation that leads to tricuspid regurgitation.

3. The medical device of claim 1, wherein the tricuspid annular constraint component comprises:
(a) an annular support element configured to maintain tricuspid valve annulus geometry;
(b) a constraint mechanism configured to prevent annular dilation; and
(c) wherein the component is positioned to maintain valve leaflet mobility while preventing regurgitation.

4. The medical device of claim 1, wherein the adjustment mechanism comprises:
(a) a first adjustment component operatively connected to the right ventricular free wall support component;
(b) a second adjustment component operatively connected to the tricuspid annular constraint component;
(c) wherein the first and second adjustment components operate independently to allow separate optimization of right ventricular support and tricuspid constraint; and
(d) wherein each adjustment component is configured for intraoperative and post-operative modification.

5. The medical device of claim 4, wherein:
(a) the adjustment mechanism includes an external adjustment interface;
(b) the external adjustment interface is configured to follow a path substantially parallel to a left ventricular assist device driveline;
(c) the external adjustment interface allows modification of support parameters without additional surgical access; and
(d) wherein the adjustment interface is configured for communication with left ventricular assist device control systems.

6. The medical device of claim 2, wherein the anchoring means comprises:
(a) a plurality of biocompatible anchor elements configured for epicardial attachment;
(b) wherein the anchor elements are distributed across the right ventricular free wall to provide stable long-term fixation;
(c) attachment points positioned to avoid interference with coronary vessels; and
(d) wherein the anchoring system maintains device position during cardiac cycles and left ventricular assist device operation.

7. The medical device of claim 1, wherein the biocompatible epicardial support structure comprises:
(a) energy-return materials configured to flex under cardiac load and return energy to ventricular tissue;
(b) conforming materials configured to adapt to individual patient cardiac anatomy;
(c) biocompatible surface coatings suitable for long-term epicardial contact; and
(d) wherein the materials provide mechanical support while assisting ventricular function through energy return mechanisms.

8. The medical device of claim 3, wherein the tricuspid annular constraint component further comprises:
(a) a commissure protection element positioned at the anterior-posterior leaflet commissure;
(b) wherein the protection element is accessible from the epicardial surface;
(c) a rigid member configured to protect commissure integrity during right ventricular dilation; and
(d) wherein the commissure protection prevents the specific leaflet separation that leads to tricuspid regurgitation in LVAD patients.

9. A method for preventing LVAD-induced right heart failure, comprising:
(a) identifying a patient requiring left ventricular assist device implantation for advanced heart failure;
(b) concurrently placing an epicardial support device configured to independently support both right ventricular free wall and tricuspid valve structures during the left ventricular assist device implantation procedure;
(c) adjusting right ventricular free wall support parameters to optimize right ventricular geometry and prevent dilation;
(d) independently adjusting tricuspid annular constraint parameters to maintain valve geometry and prevent regurgitation;
(e) securing the epicardial support device for long-term stability without requiring additional surgical access beyond routine sternotomy; and
(f) whereby both mechanical failure modes of post-LVAD right heart failure are addressed prophylactically rather than reactively.

10. The method of claim 9, wherein the epicardial support device is placed and optimized in a two-phase process comprising:
(a) performing initial placement of the epicardial support device during routine left ventricular assist device sternotomy;
(b) securing the device on right ventricular cardiac tissue;
(c) performing preliminary optimization of right ventricular free wall support and tricuspid annular constraint under baseline conditions;
(d) completing left ventricular assist device implantation; and
(e) upon activation of the left ventricular assist device, assessing right heart geometry and hemodynamic changes to initiate phase-two optimization.

11. The method of claim 9, wherein the prophylactic approach comprises:
(a) placing the epicardial support device before onset of right heart failure symptoms;
(b) providing proactive protection against right ventricular dilation during the critical period following left ventricular assist device activation;
(c) preventing the mechanical overload conditions that lead to late right heart failure in LVAD patients; and
(d) wherein the prophylactic placement addresses the unpredictability of right heart failure development with current screening tools having only 60% positive predictive value.

12. The method of claim 10, wherein phase-two optimization comprises:
(a) observing right ventricular geometric and valve changes induced by septal shift and increased preload following left ventricular assist device activation;
(b) independently adjusting right ventricular free wall support to counter dilation and geometric distortion;
(c) independently adjusting tricuspid annular constraint to maintain valve competency under altered loading conditions; and
(d) continuing iterative adjustment until stable right heart function is achieved with the left ventricular assist device in operation.

13. The method of claim 12, wherein post-LVAD activation optimization is performed intraoperatively in response to real-time hemodynamic monitoring, comprising:
(a) monitoring right ventricular geometry and valve performance with imaging and pressure measurements;
(b) identifying septal shift effects and increased right ventricular preload;
(c) modifying free wall and annular support parameters based on observed changes; and
(d) confirming optimization through hemodynamic improvement and valve integrity.

14. The medical device of claim 1, further configured for dual-phase optimization, wherein:
(a) the adjustment mechanism allows preliminary intraoperative adjustment during initial placement;
(b) independent adjustment of right ventricular free wall support and tricuspid annular constraint is enabled upon left ventricular assist device activation;
(c) both support components are configured for real-time adaptation in response to LVAD-induced septal shift and preload changes;
(d) wherein coordinated adjustment provides stable right heart geometry and valve function during LVAD support.

15. The method of claim 9, wherein the epicardial support device is utilized during cardiac surgical procedures that increase risk of right heart failure, comprising:
(a) identifying a patient undergoing cardiac surgery for mitral valve repair or replacement, coronary artery bypass grafting, aortic valve repair or replacement, or combined procedures;
(b) placing the epicardial support device on the right ventricular cardiac tissue during the surgical procedure;
(c) optimizing ventricular free wall support and tricuspid annular constraint in response to observed or anticipated changes in right heart preload and afterload resulting from the surgical intervention;
(d) independently adjusting device support parameters intraoperatively and postoperatively to maintain stable right heart geometry and valve competency under altered hemodynamic conditions; and
(e) achieving prevention or mitigation of right heart failure associated with major cardiac surgery.

16. The medical device of claim 6, wherein the anchoring means further comprises:
(a) anchoring elements configured for atraumatic removal without blood loss;
(b) repositioning capability allowing intraoperative device adjustment without tissue damage;
(c) wherein the anchoring system maintains secure fixation during normal operation while permitting controlled removal when clinically indicated; and
(d) wherein removal and repositioning can be performed without compromising cardiac tissue integrity or hemostatic control.
